(19) United States
(12) Patent Application Publication
(10) Pub. No.: US 2021/0000406 A1
(43) Pub. Date: Jan. 7, 2021

(54) Individualized Heat Acclimation Tool and Method

(71) Applicants: U.S. GOVERNMENT AS REPRESENTED BY THE SECRETARY OF THE ARMY, Fort Detrick, MD (US); The Secretary of State for Defence, London (GB)

(72) Inventors: Mark J. Buller, Douglas, MA (US); Simon K. Delves, Alverstoke (GB)

(73) Assignees: The Government of the United States, as Represented by the Secretary of the Army, Fort Detrick, MD (US); The Secretary of State for Defence, London (GB)

(21) Appl. No.: 16/767,970

(22) PCT Filed: Nov. 28, 2018
(86) PCT No.: PCT/US2018/062916

§ 371 Date: May 28, 2020

Related U.S. Application Data
(60) Provisional application No. 62/591,522, filed on Nov. 28, 2017.
(60) Provisional application No. 62/595,717, filed on Dec. 7, 2017.

(51) Int. Cl. A61B 5/22; A61B 5/01; A61B 5/024; A61B 5/11; A61B 5/0402; A61B 5/00

(52) U.S. Cl. CPC A61B 5/222; A61B 5/015; A61B 5/024; A61B 5/1112; A61B 2505/09; A61B 5/1102; A61B 5/681; A61B 5/742; A61B 5/0402

(57) ABSTRACT

A system or a method for assisting an individual to acclimatize to hot environment prior to being exposed to the hot environment using a measured heart rate with or without a measured skin temperature and/or a measured body core temperature. A Physiological Strain Index (PSI) or adaptive PSI (aPSI) is calculated for the individual to provide a target for the individual's exertion level such that an area under the calculated PSI/aPSI curve is used to determine the amount of heat acclimatization that has occurred for that particular training session and/or prior training sessions.

The patent application claims priority to and the benefit of U.S. patent application Ser. No. 62/591,522 filed on Nov. 28, 2017 and U.S. patent application Ser. No. 62/595,717 filed on Dec. 7, 2017, which are hereby incorporated by reference.

### I. FIELD OF THE INVENTION

The invention in at least one embodiment relates to a method and/or system for assisting an individual to acclimate to heat prior to being exposed to the hot environment through training at a higher Physiological Strain Index (PSI) or adaptive PSI (aPSI).

### II. BACKGROUND OF THE INVENTION

Acclimatization to hot environments will improve sporting and occupational performance in those environments upon arrival. Acclimatization occurs with repeated daily exposure to either a hot-dry or a hot-humid environment, which is sufficient to raise an individual's body (core) temperature initiating physiological responses to dissipate body heat—including a moderate to high sweating response. As a result, physical work (exercise) performance during subsequent heat exposure is enhanced due to adapted physiological response, where the body is more capable of dealing with the increased heat strain. The responses associated with acclimatization to an elevated ambient temperature include an increased stroke volume, reduced heart rate, reduced core and skin temperature, increased sweat rate, more dilute sweat (specifically a lower sodium concentration) and better maintenance of plasma volume and peripheral blood flow compared with exercising at the same intensity in the unacclimated state. The important stimulus for this adaptive response is an elevation in core temperature, which can be achieved through a constant strain model (i.e., where body core temperature—is elevated and maintained at a relatively constant body core temperature).

It is widely accepted that the constant strain, controlled hyperthermia or isothermic model of acclimatization provides the appropriate forcing function for core temperature that promotes acclimatization to hot environments. See Taylor, “Human Heat Adaptation,” Comprehensive Physiology, vol. 4, pp. 325-365, Jan. 10, 2014, abstract.

### III. SUMMARY OF THE INVENTION

According to at least one embodiment of the invention, there is a system for providing acclimatization guidance to an individual, the system including: a heart rate monitor; a timer; an output device; a processor in communication with the heart rate monitor, the timer, and the output device, the processor configured to receive a heart rate signal from the heart rate monitor; calculate a PSI score or an aPSI score (“aPSI score”) for the person using the received data; produce the calculated aPSI score to the output device; and checking to see if the training period is up (or finished) based on timing information from the timer; when the training period is not expired, then repeating these steps; and when the training period is expired, then calculating an area under the curve defined by the aPSI readings during the training period representing the level of acclimatization, and when one of the physiological readings is unavailable, using a previously stored value or calculating a value for the physiological reading. Further to the previous embodiment, the system further includes a temperature sensor in communication with the processor for providing a skin temperature reading to the processor. Further to the previous embodiments, the system is housed in a wearable device. In a further embodiment, the system includes a housing that holds the timer, the output device and the processor. Further to any of the previous embodiments, the output device includes at least one of a display, a speaker, and a transducer.

According to at least one embodiment of the invention, there is a system for providing acclimatization guidance to an individual, the system including: a chest strap having a heart rate monitor; a wrist-worn device having a timer; a display; an optional skin temperature sensor; a processor capable of being in wireless communication with the heart rate monitor and in electrical communication with the timer, the skin temperature sensor, and the display, the processor configured to receive a heart rate signal from the heart rate sensor; optionally receive a skin temperature reading from the skin temperature sensor; calculate a PSI score or an aPSI score (“aPSI score”) for the person using the received data; produce the calculated aPSI score to the display; and checking to see if the training period is up (or finished) based on timing information from the timer; when the training period is not expired, then repeating these steps; and when the training period is expired, then calculating an area under the curve defined by the aPSI readings during the training period representing the level of acclimatization.

According to at least one embodiment of the invention, there is a wrist-worn system for providing acclimatization guidance to an individual, the system including: a pulse oximeter sensor capable of contact with the individual's skin; a timer; a display; an optional skin temperature sensor capable of contact with the individual's skin; a processor in electrical communication with the pulse oximeter sensor, the timer, the skin temperature sensor, and the display, the processor configured to receive a heart rate signal from the pulse oximeter sensor; optionally receive a skin temperature reading from the skin temperature sensor; calculate a PSI score or an aPSI score (“aPSI score”) for the person using the received data; produce the calculated aPSI score to the display; and checking to see if the training period is up (or finished) based on timing information from the timer; when the training period is not expired, then repeating these steps; and when the training period is expired, then calculating an area under the curve defined by the aPSI readings during the training period representing the level of acclimatization.

Further to any of the previous embodiments, the processor is configured to receive a body core temperature from an internal temperature sensor or determine a body core temperature based at least on the heart rate from the heart rate monitor. In at least one embodiment, the internal temperature sensor adapted to be in the individual and in wireless communication with the processor to provide a body core temperature for the individual, where the body core temperature and the heart rate are used to determine the PSI.

Further to any of the previous embodiments, the system further includes an accelerometer in communication with the processor; and wherein the processor is configured to detect at least one of a resting heart rate and a resting skin temperature of the individual when a plurality of signals from the accelerometer remained below a predetermined threshold for a predetermined time period and/or substantially remained below the predetermined threshold for the predetermined time period, and the processor further configured to determine a resting body core temperature for the individual based on the resting heart rate.

Further to any of the previous embodiments, the system includes an activity completion module in communication with the processor and the processor configured to provide pacing information. In a further embodiment, the activity completion module is selected from a group consisting of a pedometer, an accelerometer tracking distance travel, a bicycle computer tracking cycling distance, and an odometer tracking cycling distance; or the activity completion module includes at least one of a pedometer, an accelerometer tracking distance travel, a bicycle computer tracking cycling distance, an odometer tracking cycling distance, or a Global Positioning System. Further to the any of the embodiments in the previous paragraphs, the system further includes an input for receiving identification of the activity being performed by the individual.

Further to any of the previous embodiments, the system further includes an alarm in communication with the processor. In a further embodiment, the processor is configured to produce an alert signal to the alarm when the calculated aPSI score exceeds a predetermined aPSI score threshold.

Further to any of the previous non-housing embodiments, the system further includes a housing that holds the timer and the processor.

Further to any of the previous embodiments, the heart rate monitor and/or the output device communicates with the processor wirelessly. Further to any of the previous embodiments, the heart rate monitor is selected from a group consisting of a heart rate sensor attached to the subject person, an EKG processor for receiving EKG signals from electrodes attached to the person, a pulse oximeter sensor, or a cardiogram processor for receiving a ballistic-cardiogram signal.

In at least one embodiment of the invention, there is a method for generating an adaptive physiological strain index (PSI/aPSI) from a skin temperature and heart rate for an individual, the method including: receiving by a processor a heart rate signal from a heart rate sensor detecting the heart rate of the individual; receiving by the processor a skin temperature reading from a temperature sensor detecting the skin temperature of the individual; calculating with the processor a body core temperature for the individual based on the heart rate signal; calculating with the processor a temperature gradient between the skin temperature reading and the body core temperature; calculating with the processor an PSI/aPSI score for the individual using the body core temperature, the temperature gradient and the heart rate signal; producing the calculated PSI/aPSI score from the processor; and checking to see if the training period is up (or finished) based on timing information from a timer when the training period is not expired, then repeating these steps, and when the training period is expired, then calculating an area under the curve defined by the PSI/aPSI readings during the training period representing the level of acclimatization. In a further embodiment, the skin temperature is not measured or detected, but instead is estimated based on the body core temperature or not used.

In at least one embodiment of the invention, there is a method for generating an adaptive physiological strain index (PSI/aPSI) for an individual, the method including: receiving by a processor a heart rate signal from a heart rate sensor detecting a heart rate of the individual; calculating with the processor a body core temperature for the individual based on the heart rate signal; calculating with the processor an PSI/aPSI score for the individual using the body core temperature and the heart rate signal; producing the calculated PSI/aPSI score from the processor; and checking to see if the training period is up (or finished) based on timing information from a timer when the training period is not expired, then repeating these steps, and when the training period is expired, then calculating an area under the curve defined by the PSI/aPSI readings during the training period representing the level of acclimatization. In a further embodiment, the method further including estimating a skin temperature based on either the body core temperature or a temperature gradient with the body core temperature.

In at least one embodiment of the invention, there is a method for generating an adaptive physiological strain index (PSI/aPSI) from a skin temperature and heart rate for an individual using a wrist-worn device having a processor and a temperature sensor, the method including: receiving by the processor a heart rate signal from a heart rate sensor detecting the heart rate of the individual; receiving by the processor a skin temperature reading from the temperature sensor detecting the skin temperature of the individual; calculating with the processor a body core temperature for the individual based on the heart rate signal; calculating with the processor a temperature gradient between the skin temperature reading and the body core temperature; calculating with the processor an PSI/aPSI score for the individual using the body core temperature, the temperature gradient and the heart rate signal; producing the calculated PSI/aPSI score from the processor; and checking to see if the training period is up based on timing information from a timer when the training period is not expired, then repeating these steps, and when the training period is expired, then calculating an area under the curve defined by the PSI/aPSI readings during the training period representing the level of acclimatization.

Further to any of the previous embodiments, the aPSI is calculated based on one of the following approaches: 1) receiving and/or storing the individual's age and using the following equation:

$$aPSI = 5 ( \frac{C {T}_{t} - C {T}_{r e s t}}{C {T}_{critical} - C {T}_{r e s t}} ) + 5 ( \frac{H {R}_{t} - H {R}_{r e s t}}{H {R}_{critical} - H {R}_{r e s t}} )$$

$$H {R}_{critical} = 0.90 ( 220 - age )$$

$$C {T}_{critical} = 39.5 ° C . + \frac{( C {T}_{t} - S {T}_{t} ) - 4}{4}$$

where CTₜ is the body core temperature at a time t, CTᵣₑₛₜ is the body core temperature at rest, HRₜ is the heart rate at a time t, HRᵣₑₛₜ is the heart rate at rest, HR_{critical} is a maximum heart rate, CT_{critical} is a maximum body temperature, and STₜ is the skin temperature; 2) calculating the PSI/aPSI score is based on the processor calculating the PSI/aPSI score based on the following equation:

$$aPSI = 5 ( \frac{C {T}_{t} - C {T}_{r e s t}}{C {T}_{critical} - C {T}_{r e s t}} ) + 5 ( \frac{H {R}_{t} - H {R}_{r e s t}}{H {R}_{critical} - H {R}_{r e s t}} )$$

where CTₜ is a body core temperature at a time t, CTᵣₑₛₜ is a body core temperature at rest, HRₜ is the heart rate at a time t, HRᵣₑₛₜ is a heart rate at rest, HR_{critical} is a maximum heart rate, and CT_{critical} is the maximum body temperature; and 3) receiving and/or storing the individual's age and using the following equation:

$$aPSI = 5 ( \frac{C {T}_{t} - C {T}_{r e s t}}{C {T}_{critical} - C {T}_{r e s t}} ) + 5 ( \frac{H {R}_{t} - H {R}_{r e s t}}{H {R}_{critical} - H {R}_{r e s t}} )$$

$$H {R}_{critical} = 0.90 ( 220 - age )$$

where CTₜ is a body core temperature at a time t, CTᵣₑₛₜ is a body core temperature at rest, HRₜ is the heart rate at a time t, HRᵣₑₛₜ is a heart rate at rest, HR_{critical} is a maximum heart rate, and CT_{critical} is a maximum body temperature.

In a further embodiment to any of the previous embodiments, the processor uses a previously stored value or calculating a value for the physiological reading when one of the physiological readings is unavailable.

In a further embodiment to any of the previous embodiments, the aPSI score is adjusted based on at least one of a fitness level, an age, a maximum heart rate, and a resting heart rate of an individual and this includes any combination of these. In a further embodiment to any of the previous embodiments, the processor produces a new aPSI score at predetermined intervals based on 1) variances in at least one of the skin temperature and the heart rate received by the processor; or 2) at least one first aPSI score at an initial time designation of the timer and calculating a new aPSI score at predetermined time intervals as provided by the timer. In a further embodiment to any of the previous embodiments, the body core temperature is calculated using a Kalman filter or an extended Kalman filter.

In a further embodiment, the aPSI score calculated by the processor is displayed.

### IV. BRIEF DESCRIPTION OF THE DRAWINGS

FIG. 1 illustrates a method according to one embodiment of the system.

FIG. 2 illustrates a system according to at least one embodiment of the invention.

FIG. 3 illustrates a system according to at least one embodiment of the invention.

FIG. 4 illustrates a system according to at least one embodiment of the invention.

FIG. 5 illustrates a system according to at least one embodiment of the invention.

FIGS. 6A-6L illustrate examples of possible user interfaces for use in at least one embodiment of the invention.

FIG. 7 illustrates a computer program product and computer implementation according to at least one embodiment of the invention.

FIGS. 8A-8F illustrate an example of using a system built according to at least one embodiment of the invention over a six-day period of acclimation.

FIGS. 9A and 9B illustrate a comparison between using a system built according to at least one embodiment of the invention and guidance obtained from the JSP539 manual.

### V. DETAILED DESCRIPTION OF THE DRAWINGS

In at least one embodiment, a system and/or a method uses the elevation of core temperature above a pre-defined threshold and through exercise/rest, core temperature is maintained above the threshold while not allowing core temperature to further increase. The theory is that a prolonged elevation in core temperature in this manner stimulates physiological responses that will lead to heat acclimatization. See Taylor, “Human Heat Adaptation,” Comprehensive Physiology, vol. 4, pp. 325-365, Jan. 10, 2014. The system and/or the method uses the area under the curve of the actual Physiological Strain Index (PSI) or adaptive PSI (aPSI) and the target PSI to track the level of acclimation that has occurred from the training. In at least one embodiment, the number of days prior to exposure to the hot environment is used to set a schedule for the individual's training.

There are at least two different approaches to setting a PSI. There is the approach developed by Moran et al. and the aPSI approach discussed in PCT Application No. PCT/US2017/027985, published as WO2017/181195 A1 on Oct. 19, 2017, which is hereby incorporated by reference. Table 1 shows the original PSI levels and the associated levels of thermal work strain according to Moran et al. (1998):

TABLE 1

| PSI | Strain |
|---|---|
| 0 | |
| 1 | No/Little |
| 2 | |
| 3 | Low |
| 4 | |
| 5 | Moderate |
| 6 | |
| 7 | High |
| 8 | |
| 9 | Very High |
| 10 | |

It has been found in the past that it is possible to exceed a PSI of 10 under certain circumstances. The above table is relevant for at least one of the disclosed embodiments, the adaptive physiological strain index (“aPSI” or “adaptive PSI”) provides a strain index score between 0 and 10 that takes into account the conditioning of the individual, the environment they are in, and the clothing they are wearing while being monitored. Existing systems would have a marathon runner having a high PSI (e.g., 11 or 12) compared to an individual wearing a fully enclosed Hazmat suit having a lower PSI (e.g., 7.5). An observer would deem the Hazmat suit individual being under more strain than the marathon runner. In at least one embodiment, aPSI addresses this inaccuracy.

In at least one embodiment, a system and/or a method is provided to use an individual's body core temperature in connection with their heart rate and skin temperature, which can be estimated or measured, to determine their aPSI. The invention in at least one embodiment relies on a method for detecting and evaluating the aPSI of the individual with a processor having suitable programming to perform the functions discussed in this disclosure. In at least one embodiment, the relationship between the body core temperature and heart rate is a quadratic relationship that varies over a range of heart rate measurements, where in at least one embodiment the range is between 50 and 180 beats/minute, and in a further embodiment, the maximum heart rate is set to 220 minus the person's age with a corresponding quadratic relationship. In a further embodiment, the system and method use a Kalman filter model or an extended Kalman filter to determine the body core temperature. In at least one embodiment, the system and method calculates (or generates) and adjusts for external factors that may influence the adaptive physiological strain index, such as the environment, clothing, physical fitness, and the person's age or weight.

The current disclosure is independent of the PSI approach used although for purposes of this disclosure the aPSI will be used as way of example.

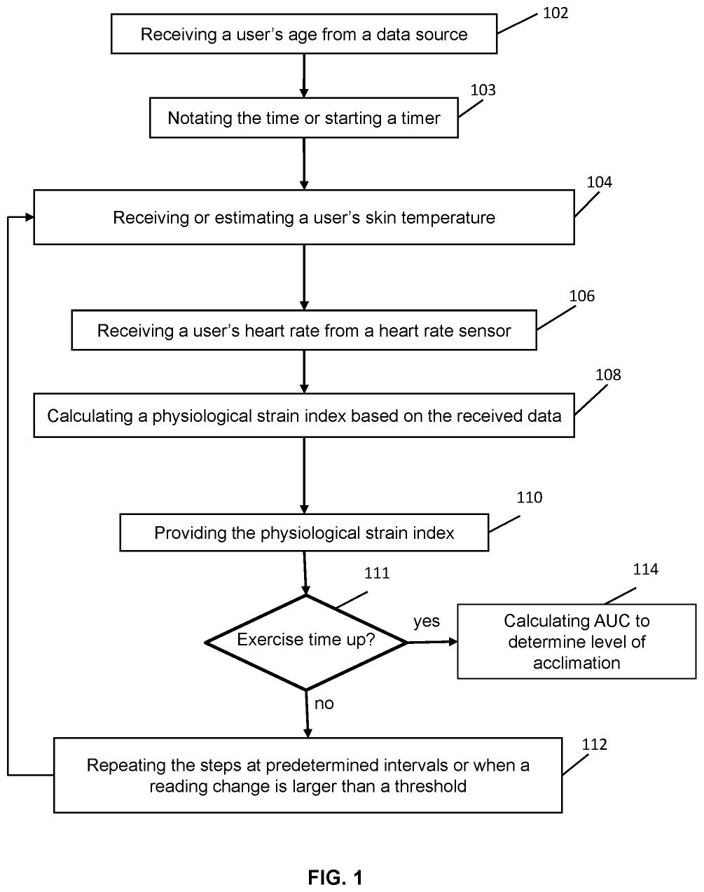

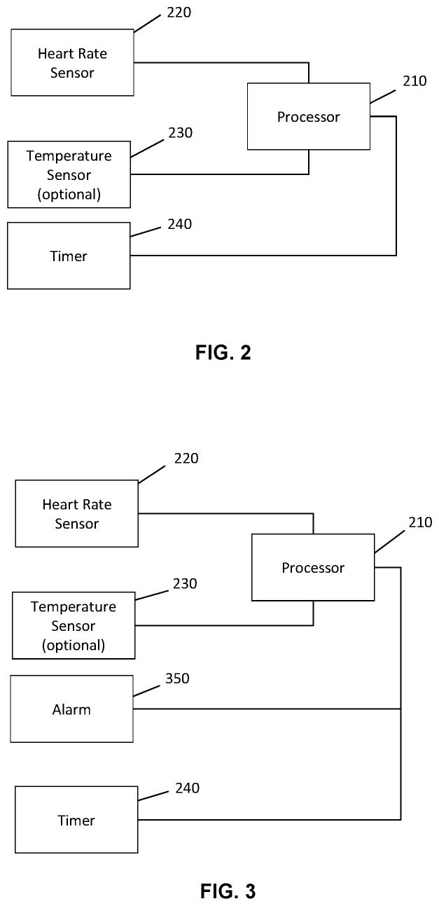

FIG. 1 illustrates a method for operation of at least one system embodiment like those illustrated in FIGS. 2-6. FIGS. 2-5 illustrate an optional temperature sensor 230 for detecting the skin temperature as part of the system. A processor 210 receives the individual's age from a data source, 102. Examples of how the individual's age can be received include: 1) the individual providing his/her age is received through an input device such as a keyboard or other data entry mechanism including virtual versions; 2) retrieval of stored data about the individual; and 3) a combination of the previous two examples. In an alternative embodiment, this step is omitted from the method. In an alternate embodiment, the resting physiological information is provided in a similar manner at least as a starting point.

The processor 210 is in electrical communication with a timer 240. The time circuit 240 is initiated and/or a time is notated, 103.

The processor 210 receives a skin temperature from at least one temperature sensor 230, 104. Or alternatively, the skin temperature is estimated from an estimated body core temperature. In a further alternative embodiment, the input of clothing that the user is wearing is used to determine a critical body core temperature instead of using the skin temperature. Either of these alternative embodiments allow omission of the temperature sensor from the system. The processor 210 receives a measured heart rate from a heart rate sensor (or heart rate monitor) 220, 106. In at least one embodiment, there is one component that provides the skin temperature and the individual's heart rate. In at least one alternative embodiment, the processor sends a request for the person's skin temperature and/or heart rate to the appropriate sensor(s) for a reading instead of a continual data feed from these sensors.

In at least one embodiment, steps 102 through 106 can be performed in a different order and/or substantially simultaneous or substantially concurrently with each other.

The processor 210 calculates the aPSI score for the individual, 108. In at least one embodiment, the aPSI score is determined based on a quadratic calculation of the values of at least one of the skin temperature (detected or estimated), the received heart rate, and the received age of the individual (which may be omitted from the calculation). In at least one embodiment, the aPSI score is calculated using a critical body core temperature that is based on a temperature gradient between the resting body core temperature, which is calculated based on the heart rate in at least one embodiment, and the skin temperature. In an embodiment using PSI, the skin temperature is not used and in a further embodiment the age is not used.

In at least one embodiment, the body core temperature is calculated in a multi-step process using an extended Kalman filter as discussed in U.S. Pat. App. Pub. No. US-2014-0180027-A1, which is hereby incorporated by reference. In a further embodiment, the processor can produce the body core temperature for the individual using a number of factors for an individual based on physical characteristics such as height, weight, and age. In at least one embodiment, using any known way to estimate a body core temperature including using any combination of skin temperature, physiological data, accelerometer data, environmental information, and clothing information. In an alternative embodiment, the core temperature is measured using an invasive approach (e.g., rectal temperature sensor) or a telemetry pill. But these alternative embodiments, are less desirable than using skin temperature and heart rate to determine the core temperature, which allows for more versatility in who may use the system.

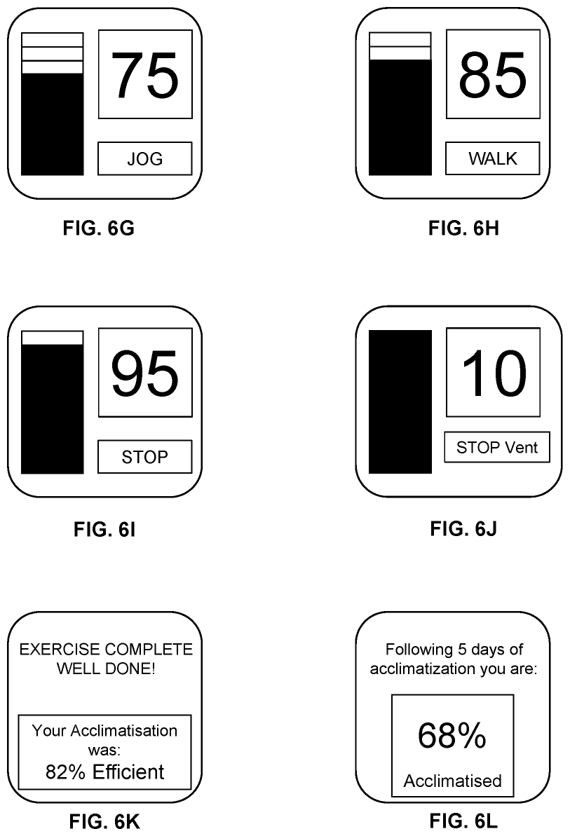

The processor 210 then provides the calculated aPSI score, 110. The calculated aPSI score may be provided to a display, a memory, a transmission system for relaying to an external device or system, and/or an alarm. The aPSI provides an improved indication of the current physiological strain of the individual being monitored, and would allow for an activity or pace change by the individual, if desired and/or possible, to lower the physiological strain. See, e.g., FIGS. 6F-6J. By providing the aPSI score to the user, the user is able to use it as a feedback loop by which exercise/thermal strain can be controlled by the individual, for example using the equation below. This allows the individual to maintain an appropriate core temperature, by modifying their work rate, which would promote the most efficient acclimatization response in the time allocated.

$$PSI = 5 ( \frac{C {T}_{t} - C {T}_{r e s t}}{39.5 - C {T}_{r e s t}} ) + 5 ( \frac{H {R}_{t} - H {R}_{r e s t}}{180 - H {R}_{r e s t}} )$$

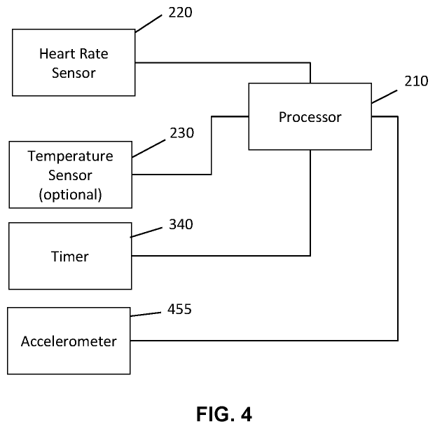

The processor 210 then inquires with the timing circuit 240 to determine whether the prescribed exercise period has been met, 111. When the exercise period has not been met, repeating the receiving (104 and 106), calculating (108), providing (110), and determining (111) steps at predetermined intervals, 112. Examples of predetermined intervals include 30 second intervals, 1 minute intervals, 2 minute intervals, 5 minute intervals, and 10 minute intervals. In a further embodiment, the method includes setting or selecting the predetermined interval prior to calculating the aPSI score. In at least one further embodiment, a timer (or timer circuit or timing circuit or clock including simulated versions) 340 illustrated in FIG. 3 can be used to delay the repeat cycle after calculating each aPSI score. In at least one embodiment, the aPSI score is calculated at variable times based on a change in the detected or estimated skin and/or body core temperature, the heart rate that exceeds a predetermined threshold, the rate of change at least one physiological signal (e.g., skin temperature or heart rate) over a predetermined change time, time left in the exercise period, or a combination of these.

When the exercise (or training) period has been met, then calculating the area under the curve (AUC) to determine the level of acclimation that has occurred during this exercise/training period, 114. The calculation of the AUC of the acclimatization PSI response for the duration of the acute exercise period provides a daily cumulative optimal dose of acclimatization which when summed over a number of days provides a dose response that can be used to define the percentage of acclimatization an individual has achieved. The equation

Area=∫ₐ^{b}f(x)dx

provides one framework in which AUC can be calculated. The AUC of the component parts of the PSI (core temperature and heart rate) may also provide further granularity to the acclimatization experience of the individual and allow for a model that better explains the cumulative acclimatization response. This could be specific to the acute acclimatization exercise or if worn throughout waking hours would provide a cumulative acclimatization response above a pre-defined threshold. This cumulative response would take into consideration the background acclimatization exposure in addition to the specific acute acclimatization exercise. In a further embodiment, this is compared with the total potential modelled dose for the timeframe in order to calculate a daily percentage of acclimatization attained. In at least one embodiment, the individual acclimatization dosimeter calculation of cumulative AUC is made and displayed as a percentage of acclimatization attained over repeated days of exercise, which allows, for example, a coach or military commander to see the progression of the individual(s)'s acclimatization progress. In an alternative embodiment, the AUC is monitored during the session, for example to show the user that acclimation progress is occurring.

In at least one further embodiment, when the aPSI score exceeds a predetermined alarm threshold, an alert is generated by an alarm 350 of FIG. 3. In at least one embodiment, the processor 210 provides an alarm signal to the alarm 350 that triggers the alert.

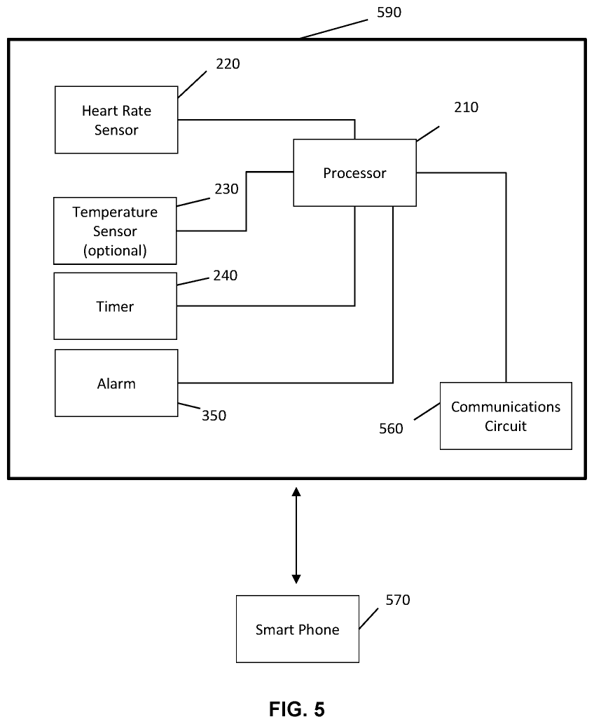

In a further embodiment illustrated in FIG. 4, the system includes at least one accelerometer 455 to monitor activity of the individual to facilitate obtaining resting physiological information about the individual such as resting body core temperature (CTᵣₑₛₜ) and resting heart rate (HRᵣₑₛₜ) and in other embodiments, the skin temperature. The illustrated system also includes the timer 240. The processor 210 is in electrical communication with the timer 240 and the accelerometer 455 to detect when the accelerometer 455 signal(s) are below a predetermined threshold for a predetermined time based on a time signal from the timer 240.

In at least one embodiment, when the accelerometer signal(s) decreases below the predetermined threshold, the processor 210 stores the current time data in memory for later comparison or alternatively begins a counter that is incremented based on the time signal. Under the comparison embodiment, when the current time data is greater than the stored time data by the predetermined time, the processor 210 pulls and/or processes the signal from the heart rate sensor 220 to obtain the resting heart rate, which then is used to determine the resting body core temperature. Under the counter embodiment, the processor 210 increments the counter based on the time signal until it matches and/or exceeds the predetermined time before pulling and/or processing the signal from the heart rate sensor 220.

In a further embodiment, when the accelerometer signal(s) exceeds the predetermined threshold momentarily before decreasing below, the time does not reset. In such a situation, the accelerometer signal(s) has substantially remained below the predetermined threshold.

In at least one embodiment, the predetermined time is 20 minutes, 25 minutes, 30 minutes, 35 minutes, etc. In an alternative embodiment, the predetermined time is shorter such as 10 minutes or 15 minutes, and the processor 210 compares the heart rate signal starting at the predetermined time to follow-on recordings while the accelerometer signal(s) remains below the predetermined threshold to determine whether the heart rate signal has stabilized. Stabilized as used in this disclosure means that the signal level falls within a range set in the processor 210 for the physiological characteristic being monitored.

Using any of the previously mentioned variables, the modified and adaptive PSI includes an ability for application to adapt to different populations, different work, diverse age ranges, and/or protective clothing environments. The equation for the adaptive PSI score in at least one embodiment is as follows:

$$aPSI = 5 ( \frac{C {T}_{t} - C {T}_{r e s t}}{C {T}_{critical} - C {T}_{r e s t}} ) + 5 ( \frac{H {R}_{t} - H {R}_{r e s t}}{H {R}_{critical} - H {R}_{r e s t}} )$$

$$H {R}_{critical} = 0.90 ( 220 - age )$$

$$C {T}_{critical} = 39.5 + \frac{( C {T}_{t} - S {T}_{t} ) - 4}{4}$$

In the adaptive PSI equation, the CTₜ is the body core temperature at a time t, CTᵣₑₛₜ is the body core temperature at rest, HRₜ is heart rate at a time t, and the HRᵣₑₛₜ is heart rate at rest, the HR_{critical} is a critical maximal heart rate threshold used to determine a maximal aPSI. In at least one embodiment the HR_{critical} in the adaptive PSI equation has a value as 90% of HRₘₐₓ as suggested by the American College of Sports Medicine Guidelines (America College 1991) and also includes the variable (220-age) for the HR_{critical} value to be configured to apply to individuals of any age. In an alternate embodiment, the HR_{critical} can be set as 90% of HRₘₐₓ derived from a VO2 max test. In at least one embodiment, the HR_{critical} is determined for the particular person based on previous physiological measurements.

The adaptive PSI equation also includes CT_{critical} as the critical body core temperature which is adapted in real-time based on a body core temperature (CTₜ), a skin temperature (STₜ), and a critical temperature such as 39.5° C. During activity the CT_{critical} will vary based on a temperature gradient between the current core temperature and the current skin temperature.

In at least one embodiment, the method and system are able to adapt to constraints on available physiological data for use.

In the case of resting body core temperature and resting heart rate, the values used may be preset, entered by the individual or another person as discussed previously, or based on physiological measurements taken at rest by the system. When the resting body core temperature is not available, then it may be set to 37.1° C. or calculated from the resting heart rate using, for example, a Kalman filter or an extended Kalman filter or other similar estimation for body core temperature based on heart rate. When the resting heart rate is not available, then it may be set at 71 beats per minute. In at least one embodiment, the system is prompted to take the current heart rate by a user or the individual to establish the individual's resting heart rate.

In at least one embodiment, the critical heart rate (HR_{critical}) is set to 180 beats per minute. In other embodiments, it is set based on the individual's age using the equation above or is obtained from another source for this specific individual based on physiological testing.

When the skin temperature is unavailable, the skin temperature is set to body core temperature minus four degrees Celsius in at least one embodiment. In at least one further embodiment, the critical body core temperature (CT_{critical}) is set based on the clothing being worn by the individual. In a further embodiment, CT_{critical} is set as follows:

TABLE 2
CT_{critical} Values based on Clothing

| Full Encapsulation in PPE | about 38.5° | C. or less |
|---|---|---|
| Long Sleeves and Pants | about 39.5° | C. or less |
| Shorts and T-shirt | about 40.0° | C. |
| Default Setting | 39.5° | C. |

As discussed above, CT_{critical} may be set pursuant to the equation above when the resting body core temperature and the skin temperature are known. In a further embodiment, the skin temperature is modified based on the location of the sensor used to obtain the skin temperature to take into account the gradient that is present on an individual's skin based on body location.

In other embodiments, where just the heart rate is available for the individual (for example, if the skin temperature sensor is omitted or not providing data), the body core temperature is calculated from the heart rate and CT_{critical} is set to 39.5° C. When the heart rate and the skin temperature are available for the individual, using the equations above and calculating body core temperature from the heart rate. When the embodiment also includes a sensor for body core temperature, then using heart rate and body core temperature to determine the aPSI and taking into account whether skin temperature is available or not and adjusting accordingly using the above-described approaches.

In at least one embodiment as illustrated, for example in FIG. 2, the methods discussed in connection with FIG. 1 are performed on the processor 210 running code that enables the performance of at least one method embodiment and is in communication with the heart rate sensor 220 and the temperature sensor 230, which is optional. Examples of a heart rate sensor 220 include a heart rate monitor attached to the individual, a processor for receiving EKG signals from electrodes attached to the person, a processor for receiving a photolthysmogram signal (e.g., a pulse oximeter), or a processor for receiving a ballistic-cardiogram signal. The processor used as part of the heart rate sensor 220 in at least one embodiment is the processor 210. Examples of a temperature sensor 230 configured to detect a skin temperature on the exterior of the individual being monitored, such as an expanse of skin, can include various analog and digital temperature sensors, and infrared thermometers. In at least one embodiment, there is a memory, data storage, or storage (not illustrated) in communication with the processor 210. The timer 240 can also be used for setting or scheduling the sampling times (or intervals) at which the heart rate is used to calculate the body core temperature. In an alternative embodiment, the predetermined intervals are set by a user or the individual being monitored. In a further alternative embodiment, the predetermined time period is stored in a memory or data storage, for example, on a memory chip located on the user's wrist, in a database located on a network, or is present in the code running on the processor 210. The timer 240, for example, can adjust the sampling intervals for various periods such as 30 second, 1 minute, and 5 minute intervals. The timer 240 can transmit a signal to the processor 210 notifying the processor 210 to perform at least one instruction, such as alerting the processor 210 that a time interval of one minute has occurred. For example, the timer 340 can be an integrated circuit, chip, or microchip used for timing, pulse, and/or oscillator applications, but may also include code running on the processor 210.

In at least one further embodiment to any of the embodiments as illustrated in FIG. 3, the system includes the alarm 350 or another similar component to produce an alert indicating the person being monitored has exceeded an alarm parameter threshold for the aPSI score as calculated by the processor 210. The alarm 350 can be contained within the system or in communication with the system to produce an alert. The alert can be produced in any sensory form, such as auditory output through a speaker, visual output through a display and/or light elements, and/or a vibration from a transducer, configured to alert the individual or a monitoring system that the PSI parameter threshold has been exceeded.

As illustrated in FIG. 4, in at least one further embodiment to any of the above embodiments, the system includes at least one accelerometer 455 in communication with the processor 210. The accelerometer 455 is configured to provide a signal to the processor 210 based on the individual's movements that are detected.

In a further embodiment to the above embodiments, the system includes a sensor internal to the individual being monitored to measure body core temperature. The sensor is in communication with the processor wirelessly. An example of the internal temperature sensor is a thermometer pill (Jonah Pill, Respironics, Bend, Oreg.) that would be orally ingested.

In at least one embodiment, the processor 210, the heart rate sensor 220, the optional temperature sensor 230, and/or the other described electronics, such as the timer 240, the alarm 350, or the accelerometer 455, embodied in the block diagrams of FIGS. 2-4, are housed within or attached to an apparatus worn by an individual being monitored, such as on the individual's chest, arm, or wrist, but is not limited in this regard.

One example embodiment is using a chest strap with a heart rate monitor to monitor the heart rate and a wrist-worn device configured to receive the output of the heart rate monitor and optionally outputs from a skin temperature sensor and/or a communications link to an internal body core temperature sensor. In at least one embodiment, the skin temperature sensor is built into the wrist-worn device. Depending on the particular implementation, the chest strap may send raw heart rate data to the wrist-worn device to process or in other implementations, the chest strap produces a heart rate signal that can be used by the wrist-worn device for processing. In at least one embodiment, the wrist-worn device is replaced by a phone or other processing device present on the individual being monitored.

A second example embodiment is a wrist-worn device that includes a heart rate sensor. The wrist-worn device may optionally include a skin temperature sensor and/or a communications link to an internal body core temperature sensor. The heart rate may be detected using a variety of techniques including pulse oximetry sensor whose signal is optionally placed through an algorithm that takes into account bodily movement (possibly detected by an accelerometer) to determine the heart rate.

Under either of these example embodiments, the wrist-worn device or other processing device is configured to perform the processing of at least one embodiment of the invention with, for example, a processor running suitable code.

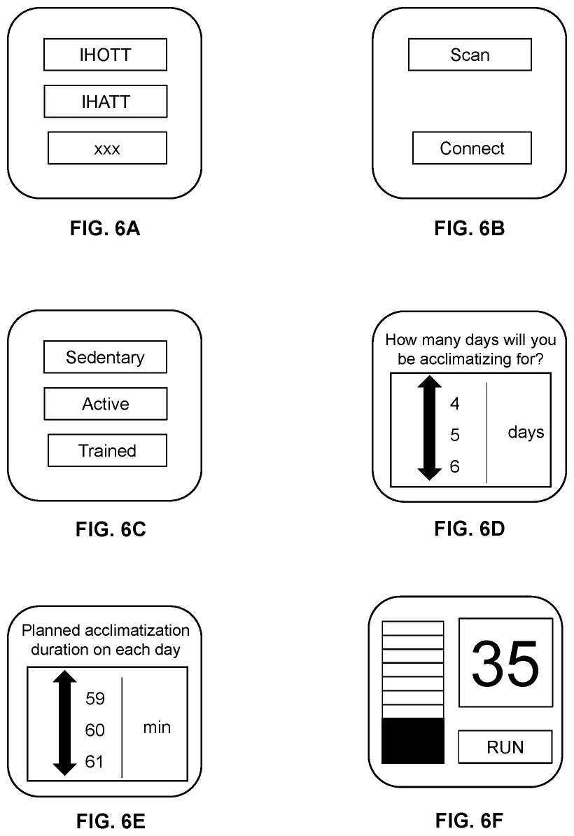

In a further embodiment illustrated in FIG. 5, the processor 210, the timer 240, the alarm 350, and the accelerometer 455 are present in one housing 590, such as that provided by a smartphone or smartwatch, in wireless communication with the heart rate sensor 220 and/or the temperature sensor 230. In a further embodiment or in addition to the previous embodiments, the body core temperature, the heart rate, and/or the aPSI score can be shown on a display present on the wearable device, such as a wrist worn display, a smart telephone, or a heads-up display, for viewing by the person being monitored.

In at least one embodiment, the processor 210 is detached from the individual being monitored and is located in external equipment such as a medical monitor, fitness tracker, smartwatch, or exercise equipment, e.g., a treadmill or bicycle, or a computer implemented device running software according to at least one method embodiment. In such an embodiment, examples of how the information is sent to such external equipment include, but is not limited to, transmitting can be sent wirelessly including optically, or by various types or arrangements of hardwire connections, or combinations thereof An example of wireless and optical transmissions is through a transmitter and a receiver. In a further embodiment to any of the previous embodiments, the information can be received through, for example, a user interface, such as a keyboard, graphical user interface (e.g. touchscreen) on a display, or a microphone.

The information and operations that are transmitted throughout the various described embodiments can be in the form of electronic data, wireless signals, or a variation thereof, for example. In at least one embodiment, the processor 210 can be designed to accomplish signal processing in the configured apparatus containing the sensors and electronics but can transmit signals to a network for further processing. In another embodiment, the processor 210 is connected to a communications circuit 560 to transmit the body core temperature, the skin temperature, the heart rate, and/or the aPSI score to an external system for monitoring and/or display. FIG. 5 illustrates a communications circuit 560 configured to communicate directly with the external system, such as the communication circuit 560 communicating directly with a smart phone 570. The information and operations that are transmitted throughout the various embodiments can be sent wirelessly, optically, or by various types or arrangements of hard wire connections, or combinations thereof, among the various system components, for example.

In a further embodiment, the system includes one or more means instead of a particular component.

A heart rate means for detecting a heart rate includes a sensor for measuring heart beats or blood flow, a heart rate sensor, a heart monitor, or another biotelemetry device configured to detect a heartbeat, heart rate, or blood flow (e.g., pulse oximetry) but is not limited in this regard and the means for measuring a heart rate or heart beat can be measured in real time or recorded for later use.

In at least one embodiment, a temperature means for measuring the skin temperature can include various manual or digital thermometer and temperature gauges, but is not limited in this regard and additional apparatuses configured to detect heat or temperature can be used. The temperature means can detect skin temperature of an area of a body, such as an area of skin, can include a manual or digital thermometer, a temperature gauge, for example but is not limited in this regard and additional apparatuses configured to detect heat or temperature of an area of a body can be used.

An input means for receiving input, such as receiving a user's age includes a user interface such as a keyboard, graphical user interface (e.g., touchscreen) on a display, or voice recognition interface but is not limited in this regard and can also include receiving data from a device, memory, database, data storage, or apparatus configured to store or transmit data.

In at least one embodiment, a calculation means for calculating an aPSI score for the person based on the detected (or estimated) skin temperature, the detected heart rate, the optionally received input age, and an optional temperature gradient between the skin temperature and a body core temperature calculated based on the detected heart rate is the processor with suitable programming to perform the steps associated with this function. In at least one embodiment, an estimated gradient between the skin temperature and the core body temperature is used based, for example and at least in part, on the clothing worn or alternatively the clothing dictates a critical core body temperature.

In an alternative embodiment, the Kalman filter model or the extended Kalman filter model is adjusted for fitness level. In particular, the aPSI score can be adjusted by increasing it for better fitness and decreasing it for lower fitness levels. In a further alternative embodiment, the Kalman filter is adjusted based on age of the person by adjusting the maximum heart rate used in the model to reflect the person's age. An example of one way to determine maximum heart rate is to use 220 minus the person's age; however, the maximum heart rate could be determined for the person based on physiological testing prior to use of the heart rate sensor. In at least one embodiment, the maximum heart rate is adjusted to reflect the heart rate for the person while leaving the starting heart rate alone and thereby adjusting the scale of the correlation between the heart rate and the body core temperature. In a further alternative embodiment, any combination of the fitness, age, resting heart rate, and maximum heart rate are used to adapt or fine-tune the aPSI score the monitored individual.

FIGS. 6A-6L illustrate an example of user interface screens that might be produced by a processor 210 running code on, for example, a wrist-worn device such as a watch or a smart phone to perform the methods disclosed in this disclosure. As such, some of the illustrated interface screens are optional and in further embodiments may be substituted by preset configuration data, for example by the coach, trainer, or commanding officer. Additionally, some screens are used to setup the parameters under which the individual will be conducting their acclimatization for heat. The user interface may be displayed on a watch, phone or specialized device and may further allow for control via touchscreen and/or buttons (physical and/or virtual).

FIG. 6A illustrates an example of an initial screen that might be used to select a particular tool to assist in the acclimatization process, and as such may be considered to be optional. IHOTT representing Intelligent Heat Optimization Training Tool. IHATT representing Intelligent Heat Acclimatization Training Tool. XXX representing a cumulative acclimatization tool. IHOTT and IHATT in at least one embodiment provide monitoring for training safety of the individual. The cumulative acclimatization tool in at least one embodiment adds together a series of training days. In at least one embodiment, one or more of these options are omitted.

FIG. 6B illustrates an example of an interface that allows the user to scan for one or more data sources (Scan) or to connect to a known data source(s) (Connect) with examples including a chest strap with a heart sensor, a heart rate sensor/monitor, and a temperature sensor (external or internal). In a further embodiment, the scan and connect buttons may result in a list being displayed from which the relevant data sources are selected. In at least one embodiment, the sensors and device with the display use wireless communication such as Bluetooth to facilitate communication. Based on this disclosure, one of ordinary skill in the art should appreciate that this interface is an example of an interface that may be omitted and accomplished by the system without user input.

FIG. 6C illustrates an interface that allows for the user to do a self-assessment for their fitness level, which in at least one embodiment allows for further customization for the particular user. Although “Sedentary,” “Active,” and “Trained” are illustrated other labels may be used or this interface may be omitted. In at least one embodiment, the button will select a particular estimated core temperature for use, for example Sedentary and Active might use a base algorithm while the Trained might use an algorithm tuned for more athletic individuals allowing, for example, different critical temperatures and/or heart rates. This interface screen in at least one embodiment is omitted. In a further embodiment, the system tunes itself to the individual based on the data collected by the system, for example, resting physiological readings. In at least one embodiment, the fitness level imparts the target a PSI score or range. For example, an active individual may have a PSI score target at 6 or 7 or a range around these points (e.g., 6-7, 5.5-6.5, 6.75-7.5, etc.). For example, a trained individual (e.g., professional athlete) may have a PSI score target of 8 or a range or proximate to 8. In an alternative embodiment, the system receives information regarding the level of medical oversight or supervision to adjust the target PSI score/range based on the level of acceptable risk for a particular acclimation process.

FIG. 6D illustrates a possible interface for the user to select the number of days in which he/she has to acclimatize to heat. One possible range of number of days is 4 days to 14 days although other ranges would be possible. The number of days allows the system to break the acclimatization into steps to provide acclimatization doses so that the goal is by the end of the number of days, the person is acclimatized for the heat. The interface could have a slide interaction, a number field or rotating dial functionality for the selection of the number of days. Alternatively, the number of days is set by someone other than the user in the case of a team of individuals striving to acclimatize for the heat.

FIG. 6E illustrates a possible interface for the user to select the length of training for each day although as with the number of days this interface may be omitted and preset by someone other than the user. In an alternative embodiment, the system will select the amount of time based on the number of days and in a further embodiment based in part on the individual's fitness level. Or a further alternative in the situation where all waking time is tracked, the amount of time is set by the system taking into account time in which the individual had an elevated PSI that simulates heat. One possible range of training for each day is between 45 minutes and 90 minutes although other ranges would be possible, but this provides for a mechanism by which the system can set the PSI for training. The interface could have a slide interaction, a number field or rotating dial functionality for the selection of the length of training.

FIGS. 6F-6I illustrate the PSI displayed as an index where PSI is multiplied by 10. These figures also illustrate information as to what the user is to be doing during the training period to reach an elevated PSI, for example approximately 7.5 which equates to a body core temperature of about 38.5 degrees Celsius. In at least one embodiment, the use of PSI reduces the error possibility that might result from directly using body core temperature. Although in an alternative embodiment, the body core temperature is used instead of PSI.

FIG. 6F illustrates the suggestion that the user “Run” or other similar activity like pedal for cycling, which may be the suggestion for a PSI between 0 and 7. Other exercise examples include, but are not limited to, circuit training, stepping or rowing whether present in a hot environment or not. FIG. 6G illustrates the suggestion that the user “Jog” or slow down, which may be the suggestion for a PSI between 7 and 8. In a further embodiment, the system will switch to the base estimated body core temperature algorithm at 7.5. FIG. 6H illustrates the suggestion that the user “Walk,” which may be the suggestion for a PSI between 8 and 9. FIGS. 61 and 6J illustrate a further safety aspect of the invention. FIG. 61 illustrates the suggestion that the user “STOP” when the PSI is between 9 and 10, and FIG. 6J illustrates the suggestion that the user “STOP Vent” when the PSI is above 10. FIG. 6J also illustrates an option where the PSI is not multiplied by 10 and is instead shown as a raw PSI number. Based on this disclosure, one of ordinary skill in the art should appreciate that the ranges and messages could be altered while staying within the scope of this invention. In an alternative embodiment, the system makes use of pacing information to pace the user through the entire time while maintaining a desired PSI level. Such a pacing approach is discussed in PCT Application No. PCT/US2017/027991, published as WO2017/181196 A1 on Oct. 19, 2017, which is hereby incorporated by reference. In such an embodiment, the system includes an activity completion module in communication with the processor and the processor configured to provide pacing information to the individual based on the time through the session, the session's goal, and the aPSI of the individual. The activity completion module may be selected from a group consisting of a pedometer, an accelerometer tracking distance travel, a bicycle computer tracking cycling distance, and an odometer tracking cycling distance; or the activity completion module includes at least one of a pedometer, an accelerometer tracking distance travel, a bicycle computer tracking cycling distance, an odometer tracking cycling distance, or a Global Positioning System.

FIG. 6K illustrates an example of how the training period completion screen might look. In at least one embodiment, the efficiency is calculated based on how the real data from the training session compared to the ideal training curve. In a further embodiment, this will take into account the warm-up period. An example of using an AUC from PSI with an initial rate of rise to 7.5 PSI within 20 minutes and maintained at 7.5 for duration of the previously inputted/set session duration may result in an efficiency score of 82%.

FIG. 6L illustrates an example of how the level of acclimatization may be displayed based on cumulative PSI AUC over previous training days and the current day using the series of session duration data. This would use the total AUC for its purposes in at least one embodiment. FIG. 6L illustrates a hypothetical where the user was 68% acclimatized after 5 days of training.

As will be appreciated by one skilled in the art based on this disclosure, aspects of the present invention may be embodied as a system, method or computer program product. Accordingly, aspects of the present invention may take the form of an entirely hardware embodiment, a processor operating with software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this disclosure, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Computer program code for carrying out operations for aspects of the present invention may be written in any combination of one or more programming languages, including an object-oriented programming language such as Xcode, Ruby, Python, Java, Smalltalk, Objective C, C++, C#, Transact-SQL, XML, or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including Bluetooth, a local area network (LAN) or a wide area network (WAN), or the connection may be made to an external computer (for example, through the Internet using an Internet Service Provider).

Aspects of the present invention are described above with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general-purpose computer, special purpose computer, or other programmable data processing apparatus to produce a machine, such that the instructions, which execute with the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

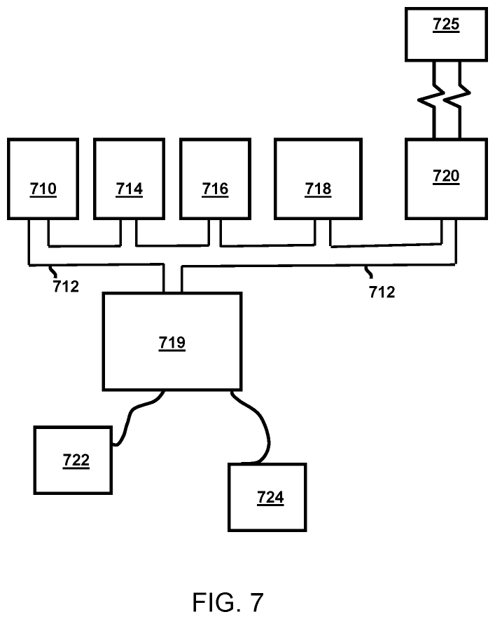

Referring now to FIG. 7, a representative hardware environment for practicing at least one embodiment of the invention is illustrated. This schematic drawing illustrates a hardware configuration of an information handling/computer system in accordance with at least one embodiment of the invention. The system includes at least one processor or central processing unit(s) (CPU) 710. The CPU(s) 710 are interconnected with system bus 712 to various devices such as a random access memory (RAM) 714, read-only memory (ROM) 716, and an input/output (I/O) adapter 718. The I/O adapter 718 can connect to peripheral devices, or other program storage devices that are readable by the system. The system can read the inventive instructions on the program storage devices and follow these instructions to execute the methodology of at least one embodiment of the invention. The system further includes a user interface adapter 719 that connects a speaker 724 and/or other user interface devices such as a touch screen device 722 to the bus 712 to gather user input. Additionally, a communication adapter 720 such as a transmitter or an antenna connects the bus 712 to a data processing network 725.

The flowchart and block diagrams in the figures illustrate the architecture, functionality, and operation of possible implementations of systems, methods and computer program products according to various embodiments of the present invention. In this regard, each block in the flowchart or block diagrams may represent a module, circuit, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the root terms “include” and/or “have”, when used in this specification, specify the presence of stated features, integers, steps, operations, elements, and/or components, but do not preclude the presence or addition of one or more other features, integers, steps, operations, elements, components, and/or groups thereof

The corresponding structures, materials, acts, and equivalents of all means plus function elements in the claims below are intended to include any structure, or material, for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention.

Although the present invention has been described in terms of particular example embodiments, it is not limited to those embodiments. The embodiments, examples, and modifications which would still be encompassed by the invention may be made by those skilled in the art, particularly in light of the foregoing teachings.

As used above “substantially,” “generally,” and other words of degree are relative modifiers intended to indicate permissible variation from the characteristic so modified. It is not intended to be limited to the absolute value or characteristic which it modifies but rather possessing more of the physical or functional characteristic than its opposite, and preferably, approaching or approximating such a physical or functional characteristic.

Those skilled in the art will appreciate that various adaptations and modifications of the embodiments described above can be configured without departing from the scope and spirit of the invention. Therefore, it is to be understood that, within the scope of the appended claims, the invention may be practiced other than as specifically described herein.

### VI. OVERVIEW

Purpose: Acclimatization to hot environments will improve sporting and occupational performance. It is widely accepted that a constant strain, controlled hyperthermia model of acclimatization provides the most appropriate forcing function for core temperature that promotes acclimatization. A prolonged elevation in core temperature in this manner stimulates the physiological responses leading to heat acclimatization. Previously, this could only be undertaken safely using direct measures of core temperature. An estimated core temperature algorithm allows for the estimation of core temperature, thus providing a safe, non-invasive approach that could be compared against an optimally modelled response by the combination of core temperature and heart rate in the calculation of the real-time physiological strain index (PSI).

Methods: Presenting the PSI in a personal display provides a real-time feedback loop by which exercise/thermal strain can be controlled by the individual. This allows the individual to maintain an appropriate core temperature, by modifying work rate, which promotes the most efficient acclimatization response in the time allocated. By way of example, a session using at least one embodiment of the present invention was used to attain a PSI of 7.0 within the first 20-30 minutes and then to maintain that PSI for a 90 minute exercise period.

Results: A calculation of the cumulative PSI acclimatization response for the duration of the acute exercise period provides a daily dose of acclimatization that can be compared against the ideal modelled response. Further, when summed over a number of days a dose response can be calculated to define the percentage of acclimatization an individual has achieved in total.

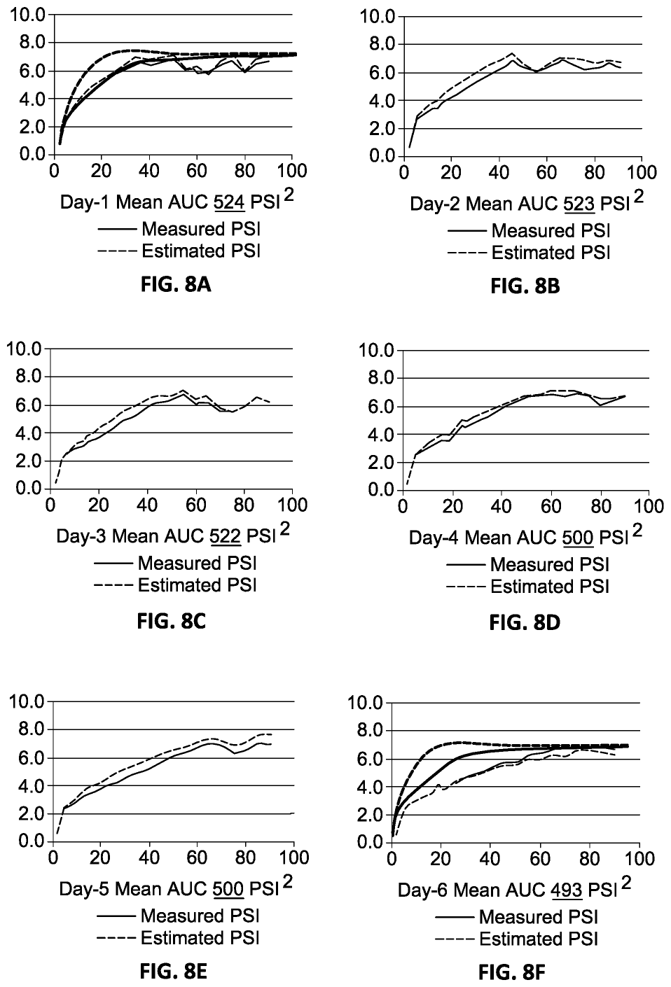

FIGS. 8A-8F illustrate the use of a system built according to at least one embodiment of the invention used over a period of six days doing stepping exercises for 90 minutes at 35 degrees Celsius and 40% relative humidity. The estimated PSI closely matched the actual measured PSI during the six days of acclimation.

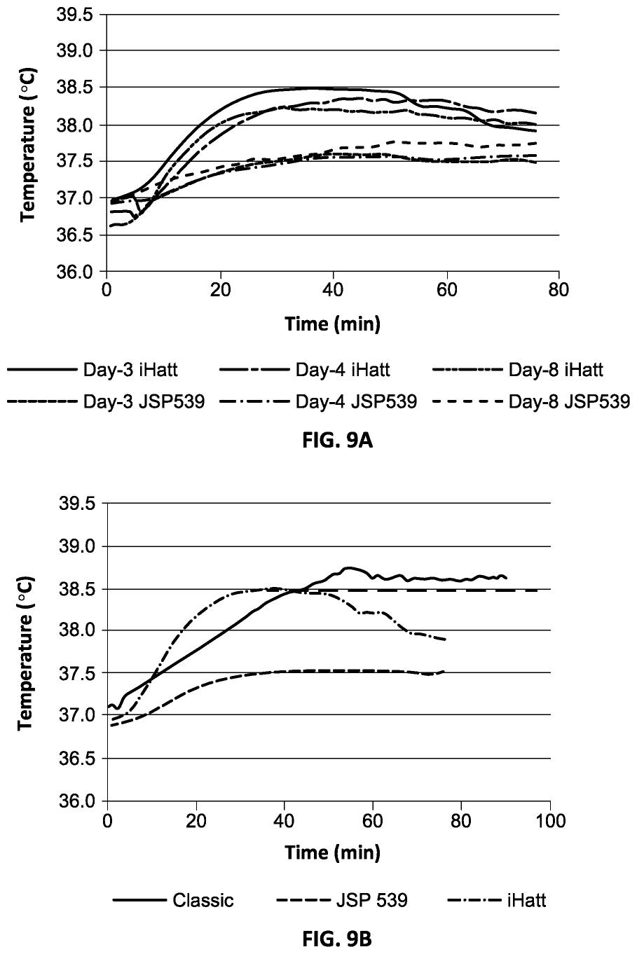

FIGS. 9A and 9B illustrate a comparison between two groups of subjects undertaking the Jungle Warfare Instructors Course at the Jungle Warfare Division (JWD). The Group 1 participants (JSP539) used the JSP 539 manual for acclimatization guidance. The Group 2 participants (iHATT) used a system built according to at least one embodiment of the invention. FIG. 9A illustrates comparisons between the two groups for Day 3, Day 4, and Day 8. FIG. 9B illustrates a comparison between the classic approach, the JSP 539 approach, and the iHATT approach with the target core body temperature being approximately 38.5 degrees Celsius as represented by the dashed line.

Conclusion: This dosimeter concept allows individuals to self-monitor thermal strain for the most efficient acclimatization exercise within the time available. This approach could be used with different exercise modalities such as running, cycling, rowing (ergometer), circuit training or stepping in hot environments.

### VII. INDUSTRIAL APPLICABILITY

In at least one embodiment, the system and/or the method will enable practical real-time monitoring systems that can improve heat acclimatization by individuals.

1. A system for providing acclimatization guidance to an individual, the system comprising:
a heart rate monitor;
a timer;
an output device;
a processor in communication with said heart rate monitor, said timer, and said output device, said processor configured to
receive a heart rate signal from said heart rate monitor;
calculate a PSI score or an aPSI score (“aPSI score”) for the person using the received data;
produce the calculated aPSI score to said output device; and
checking to see if the training period is finished based on timing information from said timer;
when the training period is not expired, then repeating these steps; and
when the training period is expired, then calculating an area under the curve defined by the aPSI readings during the training period representing the level of acclimatization, and
when one of the physiological readings is unavailable, using a previously stored value or calculating a value for the physiological reading.

2. The system according to claim 1, further comprising a temperature sensor in communication with said processor for providing a skin temperature reading to said processor.

3. (canceled)

4. (canceled)

5. (canceled)

6. The system according to claim 1, wherein said processor configured to produce a new aPSI score at predetermined intervals based on variances in at least one of the skin temperature and the heart rate received by said processor.

7. (canceled)

8. (canceled)

9. (canceled)

10. The system according to claim 1, wherein said output device includes a display in communication with said processor to display the aPSI score produced by the processor.

11. The system according to claim 1, further comprising an alarm in communication with said processor; and
said processor is configured to produce an alert signal to said alarm when the calculated aPSI score exceeds a predetermined aPSI score threshold.

12. (canceled)

13. (canceled)

14. The system according to claim 1, further comprising an activity completion module in communication with said processor and said processor configured to provide pacing information.

15. (canceled)

16. The system according to claim 14, wherein said activity completion module is selected from a group consisting of a pedometer, an accelerometer tracking distance travel, a bicycle computer tracking cycling distance, and an odometer tracking cycling distance; or
said activity completion module includes at least one of a pedometer, an accelerometer tracking distance travel, a bicycle computer tracking cycling distance, an odometer tracking cycling distance, or a Global Positioning System.

17. (canceled)

18. (canceled)

19. (canceled)

20. (canceled)

21. The system according to claim 1, wherein said heart rate monitor is selected from a group consisting of a heart rate sensor attached to the subject person, an EKG processor for receiving EKG signals from electrodes attached to the person, a pulse oximeter sensor, or a cardiogram processor for receiving a ballistic-cardiogram signal.

22. (canceled)

23. (canceled)

24. (canceled)

25. (canceled)

26. (canceled)

27. (canceled)

28. (canceled)

29. (canceled)

30. (canceled)

31. (canceled)

32. A method for providing acclimatization guidance for an individual, the method comprising:
receiving by a processor a heart rate signal from a heart rate sensor detecting a heart rate of the individual;
calculating with the processor a body core temperature for the individual based on the heart rate signal;
calculating with the processor an PSI/aPSI score for the individual using the body core temperature and the heart rate signal;
producing the calculated PSI/aPSI score from the processor; and
checking to see if the training period is finished based on timing information from a timer
when the training period is not expired, then repeating these steps, and
when the training period is expired, then calculating an area under the curve defined by the PSI/aPSI readings during the training period representing the level of acclimatization.

33. (canceled)

34. (canceled)

35. The method of claim 32, wherein calculating the PSI/aPSI score is based on said processor calculating the PSI/aPSI score based on the following equation:
$$aPSI = 5 ( \frac{C {T}_{t} - C {T}_{rest}}{C {T}_{critical} - C {T}_{rest}} ) + 5 ( \frac{H {R}_{t} - H {R}_{rest}}{H {R}_{critical} - H {R}_{rest}} )$$
where CTₜ is a body core temperature at a time t, CTᵣₑₛₜ is a body core temperature at rest, HRₜ is the heart rate at a time t, HRᵣₑₛₜ is a heart rate at rest, HR_{critical} is a maximum heart rate, and CT_{critical} is the maximum body temperature

36. (canceled)

37. (canceled)

38. (canceled)

39. (canceled)

40. The method of claim 32, further comprising adjusting by the processor the PSI/aPSI score based on at least one of a fitness level, an age, a maximum heart rate, or a resting heart rate of the individual.

41. The method of claim 32, further comprising said processor configured to produce a new PSI/aPSI score at predetermined intervals based on variances in the detected heart rate by the heart rate sensor.

42. (canceled)

43. A system for providing acclimatization guidance to an individual, the system comprising:
a heart rate monitor;
a wrist-worn device having
a timer;
a display;
a skin temperature sensor;
a processor capable of being in wireless communication with said heart rate monitor and in electrical communication with said timer, said skin temperature sensor, and said display, said processor configured to
receive a heart rate signal from said heart rate sensor;
receive a skin temperature reading from said skin temperature sensor;
generate a PSI score or an aPSI score (“aPSI score”) for the person using the received data;
produce the aPSI score to said display; and
determining whether the training period has finished based on timing information from said timer;
when the training period is not expired, then repeating these steps; and
when the training period is expired, then calculating an area under the curve defined by the aPSI readings during the training period representing the level of acclimatization.

44. The system according to claim 43, wherein the processor configured to receive a body core temperature from an internal temperature sensor or determine a body core temperature based at least on the heart rate from said heart rate monitor.

45. (canceled)

46. The system according to claim 43, wherein said wrist-worn device having an activity completion module in communication with said processor and said processor configured to provide pacing information.

47. (canceled)

48. (canceled)

49. (canceled)

50. (canceled)

51. (canceled)

52. The system according to claim 43, wherein said processor configured to generate a new aPSI score at predetermined intervals based on variances in at least one of the skin temperature and the heart rate received by said processor.

53. The system according to claim 43, wherein said processor configured to generate a new aPSI score based on calculating at least one first aPSI score at an initial time designation of said timer and calculating the new aPSI score at predetermined time intervals as provided by said timer.

54. The method of claim 32 further comprising:
receiving by the processor a skin temperature reading from a temperature sensor detecting the skin temperature of the individual;
calculating with the processor a temperature gradient between the skin temperature reading and the body core temperature; and
wherein calculating with the processor the PSI/aPSI score for the individual is based on the body core temperature, the temperature gradient and the heart rate signal.

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. The system according to claim 43, wherein the heart rate monitor is part of a chest strap.

74. The system according to claim 43, wherein the heart rate monitor is a pulse oximeter sensor, said wrist-worn device includes said pulse oximeter sensor.
